(19) United States
(12) Patent Application Publication
(10) Pub. No.: US 2014/0257359 A1
(43) Pub. Date: Sep. 11, 2014

(54) TEMPORARY SEALING DEVICE WITH BLOOD FLASHBACK FOR VESSEL LOCATION

(71) Applicant: ST. JUDE MEDICAL PUERTO RICO LLC, Caguas, PR (US)

(72) Inventors: Zachary J. Tegels, Minneapolis, MN (US); Troy T. White, Maple Grove, MN (US)

(73) Assignee: St. Jude Medical Puerto Rico LLC, Caguas, PR (US)

(21) Appl. No.: 13/794,498

(22) Filed: Mar. 11, 2013

(51) Int. Cl. A61B 17/00

(52) U.S. Cl. CPC A61B 17/0057
USPC 606/194

(57) ABSTRACT

A vessel location device includes a catheter, an expandable member, and an inflation member. The catheter includes a distal end portion, a proximal end portion and a blood flashback lumen, wherein the blood flashback lumen has a distal opening positioned at the distal end portion and a proximal opening at the proximal end portion. The expandable member is positioned at the distal end portion at a location distal of the distal opening of the blood flashback lumen. The inflation member is configured to deliver a flow of inflation fluid to the expandable member to selectively expand the expandable member within a vessel to temporarily seal a vessel puncture.

### TECHNICAL FIELD

The present disclosure relates to tissue puncture closure devices, and more specifically relates to tissue puncture closure devices having temporary sealing and blood flashback features.

### BACKGROUND

Various surgical procedures are routinely carried out intravascularly or intraluminally. For example, in the treatment of vascular disease, such as arteriosclerosis, it is a common practice to access the artery and insert an instrument (e.g., a balloon or other type of catheter) to carry out a procedure within the artery. Such procedures usually involve the percutaneous puncture of the artery so that an insertion sheath may be placed in the artery and thereafter instruments (e.g., a catheter) may pass through the sheath and to an operative position within the artery. Intravascular and intraluminal procedures unavoidably present the problem of stopping the bleeding at the percutaneous puncture after the procedure has been completed and after the instruments (and any insertion sheaths used therewith) have been removed. Bleeding from puncture sites, particularly in the case of femoral vascular punctures, may be stopped by utilizing vascular closure devices.

Typical closure devices position a sealing material adjacent to the vascular puncture along an exterior of the vessel wall. Positioning the sealing material too far proximally of the vascular puncture may lead to problems in stopping blood flow through the vascular puncture and maintaining hemostasis. In order to properly position the sealing material, the vascular puncture is first located. Puncture location may be determined in a number of ways, including, for example, positioning a vessel location device, which includes an expandable anchor member, through the vascular puncture with the expanded anchor abutting against an inner wall of the vessel. Challenges exist in properly positioning the expanded anchor relative to the vascular puncture and maintaining a position of the expanded anchor, particularly during delivery of the sealing material to the vascular puncture. Known location devices rely primarily on tactile feedback or a marker on the location device to help determine a position of the vessel location device relative to the vessel puncture.

### SUMMARY

One aspect of the present disclosure relates to a vessel location device including a catheter, an expandable member, and an inflation member. The catheter includes a distal end portion, a proximal end portion and a blood flashback lumen, wherein the blood flashback lumen has a distal opening positioned at the distal end portion and a proximal opening at the proximal end portion. The expandable member is positioned at the distal end portion at a location distal of the distal opening of the blood flashback lumen. The inflation member is configured to deliver a flow of inflation fluid to the expandable member to selectively expand the expandable member within a vessel to temporarily seal a vessel puncture.

The inflation member may include an inflation tube extending through the blood flashback lumen, the expandable member being mounted to a distal end portion of the inflation tube. The catheter and inflation member may be connected to each other at the distal end portion of the catheter. The catheter and inflation member may be connected to each other at the proximal end portion of the catheter. The catheter and inflation member may be connected to each other at a proximal bond location, and the proximal opening of the blood flashback lumen may be positioned distal of the proximal bond location.

The catheter may include a dual lumen construction having first and second lumens, wherein the blood flashback lumen includes the first lumen and the inflation member includes the second lumen. The first lumen may have a circular cross-sectional shape and the second lumen may have a non-circular cross-sectional shape. The first lumen may have a non-circular cross-sectional shape and the second lumen may have a circular cross-sectional shape. The inflation member may include an inflation lumen and the inflation member may be arranged coaxial with the blood flashback lumen.

Another aspect of the present disclosure relates to a vessel location device including a catheter and an expandable member. The catheter includes first and second lumens, a distal end portion, and a proximal end portion. The expandable member is positioned at the distal end portion and arranged in flow communication with the first lumen. The second lumen includes a distal opening arranged proximal of the expandable member at the distal end portion, and a proximal opening at the proximal end portion. The vessel location device is configured to advance through a vessel puncture and into a vessel wherein the expandable member is expanded to limit blood flow through the vessel puncture, and the second lumen provides blood flashback to indicate a position of the vessel location device relative to blood flow in the vessel.

The catheter may include first and second tubes defining the first and second lumens, respectively. The first and second tubes may be bonded to each other at the distal and proximal end portions of the catheter. The second tube may extend through the first lumen. The expandable member may include an inflation balloon having a distal waist and a proximal waist, wherein the inflation balloon is connected to the catheter at a distal bond formed between the proximal waist and the first and second tubes. The first and second tubes may be connected together at a proximal bond, and the proximal opening of the second lumen may be positioned distal of the proximal bond. The vessel location device may include a support core wire extending through one of the first and second lumens to the distal end portion. The catheter may include an integrally formed, dual lumen construction.

A further aspect of the present disclosure relates to a method of locating a vessel. The method includes providing a catheter having a distal end portion, a proximal end portion, a blood flashback lumen, and an inflation lumen, and an expandable member positioned at the distal end portion at a location distal of a distal opening of the blood flashback lumen. The method further includes positioning the expandable member through a vessel puncture, providing a flow of blood through the blood flashback lumen, inflating the expandable member via the inflation lumen, and abutting the inflated expandable member into contact with an inner surface of the vessel adjacent to the vessel puncture to temporarily seal the vessel puncture. Temporarily sealing the vessel puncture stops blood flow through the blood flashback lumen.

The expandable member may include an inflation balloon having a distal waist and a proximal waist, and the catheter may include first and second tubes defining the inflation lumen and blood flashback lumen, respectively, and the method may further include providing a distal bond between the proximal waist and first and second tubes. Providing a flow of blood through the blood flashback lumen may include flowing blood into the distal opening of the blood flashback lumen, and flowing blood out of a proximal opening of the blood flashback lumen at a location distal of a proximal end of the inflation lumen. The catheter may be formed as an extruded dual lumen structure.

The foregoing and other features, utilities, and advantages of the invention will be apparent from the following detailed description of the invention with reference to the accompanying drawings.

### BRIEF DESCRIPTION OF THE DRAWINGS

FIG. 1 is a side view of an example vessel location device in accordance with the present disclosure.

FIG. 1A is a cross-sectional view of the vessel location device of FIG. 1 taken along cross-section indicators 1A-1A.

FIG. 1B is a cross-sectional view of the vessel location device of FIG. 1 taken along cross-section indicators 1B-1B.

FIG. 1C is a cross-sectional view of the vessel location device of FIG. 1 taken along cross-section indicators 1C-1C.

FIG. 1D is a cross-sectional view of the vessel location device of FIG. 1B taken along cross-section indicators 1D-1D.

FIG. 1E is a detailed view of a distal end portion of the vessel location device shown in FIG. 1D.

FIG. 2 is a cross-sectional view showing components of the vessel location device of FIG. 1 prior to forming a distal bond.

FIG. 3A is a cross-sectional view of the vessel location device of FIG. 1 showing a different distal bond configuration.

FIG. 3B is a cross-sectional view of the vessel location device of FIG. 3A taken along cross-section indicators 3B-3B.

FIG. 3C is a detailed view of a distal end portion of the vessel location device of FIG. 3A.

FIG. 4 shows components of the vessel location device of FIG. 3A prior to forming a distal bond.

FIG. 5 is a side view of another example vessel location device in accordance with the present disclosure.

FIG. 5A is a cross-sectional view of the vessel location device of FIG. 5 taken along cross-section indicators 5A-5A.

FIG. 5B is a cross-sectional view of the vessel location device of FIG. 5A taken along cross-section indicators 5B-5B.

FIG. 5C is an alternative cross-sectional view of the vessel location device of FIG. 5A taken along cross-section indicators 5A-5A.

FIGS. 6A-6G show steps of an example method of locating and sealing a vessel puncture using the vessel location device of FIG. 1.

### DETAILED DESCRIPTION

As mentioned above, vascular procedures are conducted throughout the world and require access to a vessel through a puncture. Most often, the vessel is a femoral artery. Many times a closure device is used to deliver a sealing plug to close the puncture following completion of the procedure. However, sometimes the sealing plug is difficult to position against an exterior situs of the arteriotomy. The plug and anchor need to seat properly against the arteriotomy to close the vascular access opening. The present disclosure describes methods and apparatuses that facilitate locating a vessel puncture intravascularly using an expandable anchor and providing additional visual indicators to the operator of a position of the anchor relative to a flow of blood through the vessel.

While the vascular instruments shown and described below are directed primarily to vessel locating devices and puncture sealing devices, the application of principles described herein are not limited to the specific devices shown. The principles described herein may be used with any medical device. Therefore, while the description below is directed primarily to arterial procedures and certain embodiments of a vascular closure device, the methods and apparatus are only limited by the appended claims. Applications of closure devices including those implementing principles described herein include closure of a percutaneous puncture or incision in tissue separating two internal portions of a living body, such as punctures or incisions in blood vessels, ducts or lumens, gall bladders, livers, hearts, etc.

As used in this specification and the appended claims, the term “engage” and “engagable” are also used broadly to mean interlock, mesh, or contact between two devices. A “tube” is an elongated device with a passageway. The passageway may be enclosed or open (e.g., a trough). A “lumen” refers to any open space or cavity in a bodily organ, especially in a blood vessel. The words “including” and “having,” as used in the specification, including the claims, have the same meaning as the word “comprising.”

The present disclosure is directed to a vessel location device that provides a bleed indication lumen (also referred to as a blood flashback lumen) in conjunction with a balloon or other temporary sealing device. The bleed indication port provides a secondary visual indication via cessation of pulsatile blood flow from the vessel location device of a correct artery location. The visual indication may be used in addition to tactile feedback provided by the balloon via contact with a wall of the vessel.

As soon as blood begins flowing through the bleed indication lumen and is expelled at a proximal portion of the device, the operator has a visual indication that the expandable member is positioned within the vessel and knows to stop advancing the device further into the vessel, thereby reducing the chance of damaging the vessel. The bleed indication lumen may also indicate when the expandable member (e.g., balloon) has been inserted into the vessel and operated into an expanded position, and then properly positioned to stop blood flow through the vessel puncture. When no blood is flowing through the blood indication lumen, the operator has a visual indication that the expandable member has properly seated in the vessel. The operator may then adjust an amount of withdrawal force being applied to the device in order to avoid causing damage to the vessel.

An example embodiment of a vessel location device in accordance with the present disclosure may include a coaxial construction in which the inflation and blood flashback lumens are provided by two separate tubes arranged concentric with each other. The balloon inflation lumen may have a smaller diameter defined by an inflation tube, which is inserted through a larger diameter blood flashback lumen defined by a second tube. The balloon inflation lumen may have a core wire extending along at least a portion of a length thereof to provide additional support and kink resistance in the device. A balloon or other expandable member may be mounted to a distal end portion of the tube defining the balloon inflation lumen. The balloon may be positioned distal of a distal opening into the blood flashback lumen so that blood flow ceases as the balloon is abutted against the vessel wall adjacent to the vessel puncture. Providing an axial spacing between the balloon and distal opening into the blood flashback lumen may provide reduced risk of the inflated balloon getting hung up on plaque in the vessel, a vessel side branch, or other obstruction, which may result in the blood flashback lumen providing a false indication to the operator of the actual position of the balloon. Positioning the distal opening of the blood flashback lumen spaced proximal of the balloon may provide these and other advantages as compared to a design in which the distal opening of the blood flashback lumen is positioned distal of the balloon or directly adjacent to a proximal surface of the balloon.

In the dual tube design described above, the two tubes may be attached to each other at a proximal end by bonding the two tubes together to form a proximal bond. The bonding may be done using, for example, an adhesive or thermal bonding technique. A hole may be formed in the tube defining the blood flashback lumen at a location distal of the proximal bond. The hole may be used as a proximal blood flow outlet for the blood flashback lumen.

The two tubes may be attached to each other at a distal end portion of a tube defining the blood flashback lumen. The balloon inflation tube may extend distally beyond the distal end of the blood flashback tube. The two tubes may be connected to each other using various bonding methods and connections at various locations. In one example, the two tubes are connected to each other along only one side of the balloon inflation tube. In another example, the distal bond is defined in part by a proximal waist of the balloon. The distal bond may include a connection between the balloon inflation tube, the blood flashback tube and the proximal waist. The two tubes may be attached to each other at additional locations along the length of the two tubes in order to reduce the chance of buckling and kinking of the vessel location device.

In another example, the vessel location device comprises a dual lumen tube such as a dual lumen tube formed by a single extrusion. One lumen of the dual lumen tube provides a balloon inflation lumen for delivery of inflation fluid to the balloon or other expandable member at a distal end portion of the device. The other lumen may provide a blood flashback lumen. The two lumens may have any desired cross-sectional shape such as, for example, circular, semicircular, or crescent shape. The lumens may have a cross-sectional shape that varies along a length of the dual lumen tubing. A portion of the tubing having only a single lumen formed therein may extend distally beyond a distal opening into the blood flashback lumen to provide structure for mounting the balloon to the tubing.

The example vessel location devices disclosed herein may provide a secondary visual feedback mechanism for locating a vessel puncture when using a temporary sealing device. The vessel location devices may utilize blood flashback to provide the secondary visual feedback to the operator of a correct position of the device relative to the vessel puncture, which may be provided in addition to tactile feedback from the inflated balloon within the vessel. The vessel location devices include an integrated blood flashback feature, whereas other systems and methods may provide blood flashback via other mechanisms such as, for example, a separate insertion sheath. The integrated nature of the blood flashback features in a vessel location device as disclosed herein may provide immediate visual indication that the balloon has entered the vessel lumen. As such, the operator may recognize that the device does not require further insertion into the vessel and the expandable member may be expanded and pulled back into contact with the vessel to close the vessel puncture. Without the immediate visual indication provided by the blood flashback features of the present disclosure, the operator may inadvertently advance the balloon too far into the vessel lumen, which may result in undue damage to the vessel when the expandable member is expanded and pulled back along the length of the vessel lumen toward the vessel puncture. Furthermore, the cessation of blood flashback through the blood flashback lumen may provide immediate feedback to the operator that the vessel puncture has been properly located in a position that temporarily seals the vessel puncture.

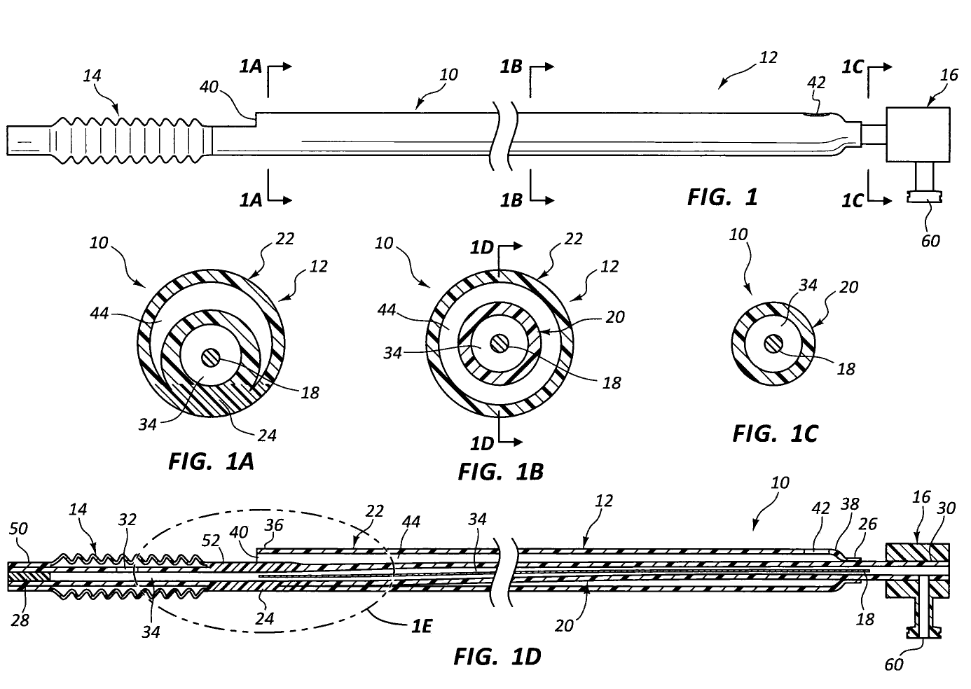

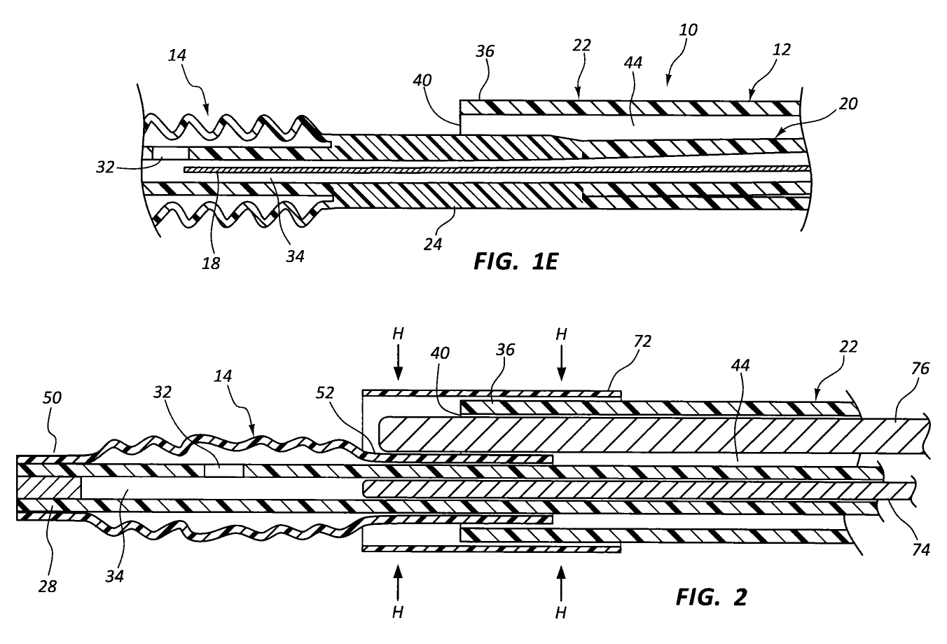

Referring now to FIGS. 1-2, an example vessel location device 10 is shown including a catheter 12, an expandable member 14, a hub 16, and a core wire 18 (see FIGS. 1A-1E). The catheter may include a plurality of lumens. The lumens may be defined by separate lengths of tubing. The tubing may be bonded to each other at a distal bond location 24. A separate proximal bond 26 may also be provided at a proximal end portion of the catheter 12. The tubing may be bonded together at other locations along a length of the catheter 12. The core wire 18 may extend through one of the lumens to provide additional stiffening and kink resistance for the catheter 12. The expandable member 14 may be positioned at a distal end portion of the catheter 12. The expandable member 14 may be positioned distal of a distal opening into a blood flashback lumen of the catheter 12. The expandable member 14 (e.g., balloon) includes distal and proximal waists 50, 52.

The catheter 12 may include an inflation tube 20 and a blood flashback tube 22. The inflation tube 20 may include distal and proximal ends 28, 30, an inflation outlet 32, and an inflation lumen 34 (see FIG. 1D). The blood flashback tube 22 may include distal and proximal ends 36, 38, a distal opening 40, a proximal opening 42, and a flashback lumen 44 (see FIG. 1D). The blood flashback tube 22 may terminate at a proximal location, which is distal of a proximal most end of the inflation tube 20. In one example, the blood flashback tube 22 terminates distal of the hub 16. The inflation lumen 34 may be connected in flow communication with an inflation port 60 of the hub 16. The inflation lumen 34 may be connected in flow communication with the expandable member 14 via the inflation outlet 32. A distal end of the inflation lumen 34 may be closed at the distal end 28 of the inflation tube 20.

The distal opening 40 of the blood flashback tube 22 may be positioned proximal of the expandable member 14. The distal opening 40 may be provided as an open end into the flashback lumen 44 and may face in a distal direction. The distal opening 40 may be defined in other ways such as, for example, forming a hole in a sidewall of the blood flashback tube 22 proximal of the distal end 36. The distal opening 40 may face in a lateral direction.

The proximal opening 42 may be positioned at the proximal end of the catheter 12 at a location such as, for example, distal of the hub 16 and distal of a proximal end 38 of the blood flashback tube 22. The proximal opening 42 may be formed by cutting or otherwise forming a hole through a sidewall of the blood flashback tube 22. In other embodiments, the proximal opening 42 may be positioned at a proximal end of the blood flashback tube 22 and may face in a proximal direction.

At least FIG. 1D shows the blood flashback tube 22 bonded to the inflation tube 20 around an entire periphery of the inflation tube 20. Other attachment arrangements are possible including, for example, connecting the distal end of the blood flashback tube 22 to the hub 16 rather than directly to the inflation tube 20. In such an example, the proximal bond 26 may be eliminated by connecting the blood flashback tube 22 directly to the hub 16 rather than directly to the inflation tube 20.

The distal bond 24 may be formed in a similar manner as the proximal bond 26 shown in FIG. 1D. For example, the distal bond 24 may be formed by connecting the blood flashback tube 22 to an outer surface of the inflation tube 20 around an entire periphery of the inflation tube 20. The distal opening 40 may be formed in a sidewall of the blood flashback tube 22 at a location proximal of the distal bond 24. In another example, as shown in FIGS. 1D and 1E, the inflation tube 20 is bonded to the blood flashback tube 22 along only a portion of the periphery of the inflation tube 20 at the distal end 36 of the blood flashback tube 22. FIG. 1A shows the distal bond 24 having a continuous, integral construction of material between the inflation tube 20 and blood flashback tube 22.

The distal bond 24 may also include a connection with the proximal waist 52 of the expandable member 14. The proximal waist 52 may be concurrently bonded to the inflation tube 20 and a portion of the blood flashback tube 22. FIG. 1A shows the distal bond 24 having a continuous, integral construction at the point of connection between the inflation tube 20, blood flashback tube 22, and proximal waist 52.

FIG. 2 shows an arrangement for forming the distal bond 24. A mandrel 74 may be inserted through the inflation lumen 34. The inflation tube 20 is positioned in the blood flashback lumen 34. A mandrel 76 may be inserted through the flashback lumen 44 along an exterior of the inflation tube 20. The proximal waist 52 of the expandable member 14 may be inserted into the flashback lumen 44 surrounding the inflation tube 20. A heat sleeve 72 may be inserted over the mandrels 74, 76, the distal end 36 of the blood flashback tube 22, the proximal waist 52, and a portion of the inflation tube 20. Heat H may be applied around an entire periphery of the heat sleeve 72. The heat H may cause the material of the inflation tube 20, blood flashback tube 22 and proximal waist 52 to flow together to form a single, integral distal bond structure as shown in FIGS. 1A and 1E. The mandrels 74, 76 and heat sleeve 72 may be removed and the distal bond 24 and distal opening 40 remain as shown in FIG. 1E. The core wire 18 may then be inserted through the inflation lumen 34, for example, into a position overlapping at least a portion of the distal bond 24 as shown in FIG. 1E.

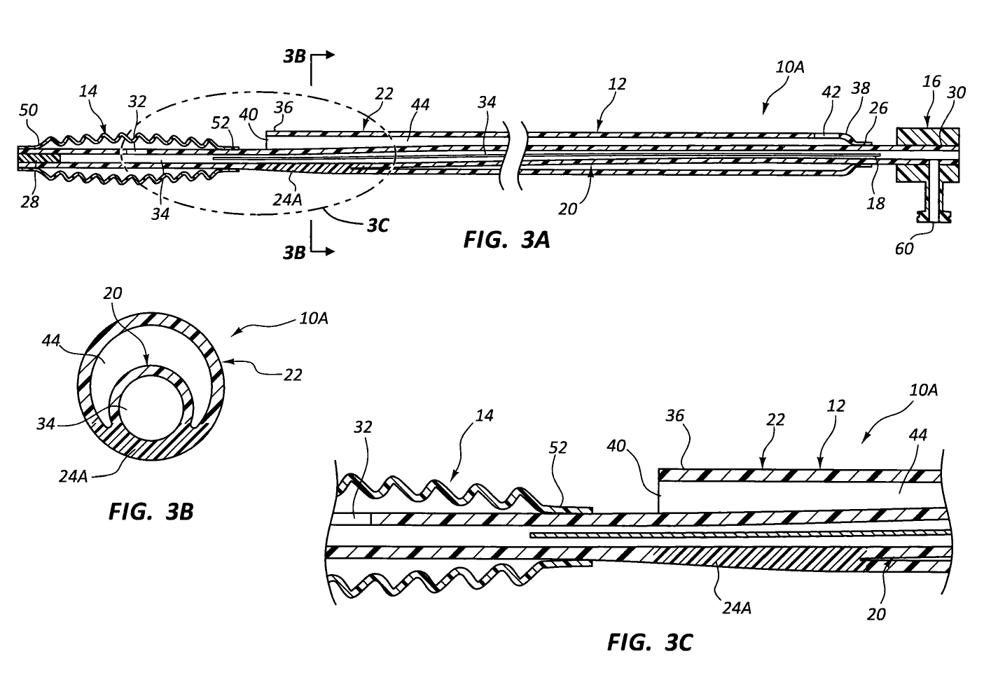

Referring to FIGS. 3A-3C, another example vessel location device 10A is shown having an alternative distal bond 24A. The distal bond 24A is formed along only a portion of the peripheral surface of the inflation tube 20 such as along only one side surface of the inflation tube 20. The distal bond 24A is shown positioned proximal of the proximal waist 52 of the expandable member 14. In other examples, at least a portion of the proximal waist 52 may be included in the distal bond 24A. FIG. 3B shows the location of the distal bond 24A along only one side of the inflation tube 20 and along only one side of an internal surface of flashback lumen 44. The distal bond 24A may be formed by applying heat H at a discrete location around a periphery of the inflation tube 20 and blood flashback tube 22.

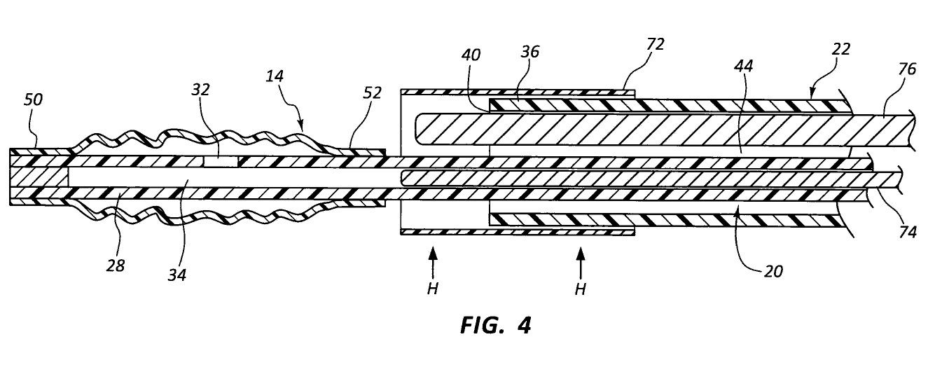

FIG. 4 shows an assembly of subcomponents of the vessel location device 10A prior to creating the distal bond 24A. A mandrel 74 is inserted through the inflation tube 20. The inflation tube 20 is positioned in the blood flashback tube 22. A mandrel 76 is inserted through the flashback lumen 44 along an exterior of the inflation tube 20. A heat sleeve is advanced over the distal end 36 of the blood flashback tube 22 and overlaps portions of the mandrels 74, 76. Heat H is applied along only one side of the assembly shown in FIG. 4 (e.g., along a length of heat sleeve 72 at a single location around a periphery of the heat sleeve 72). The discrete application of heat H creates the distal bond 24A shown in FIGS. 3A-3C along only one side of the inflation tube 20.

The expandable member 14 may be mounted to the inflation tube 20 in bonding steps separate from formation of the distal bond 24A. The heat H may be applied in any number of ways including, for example, a hot jaw, laser welding, or ultrasound. In other examples, the bonding between inflation tube 20 and blood flashback tube 22 may occur using other bonding techniques besides application of heat including, for example, an adhesive or a sonic welding.

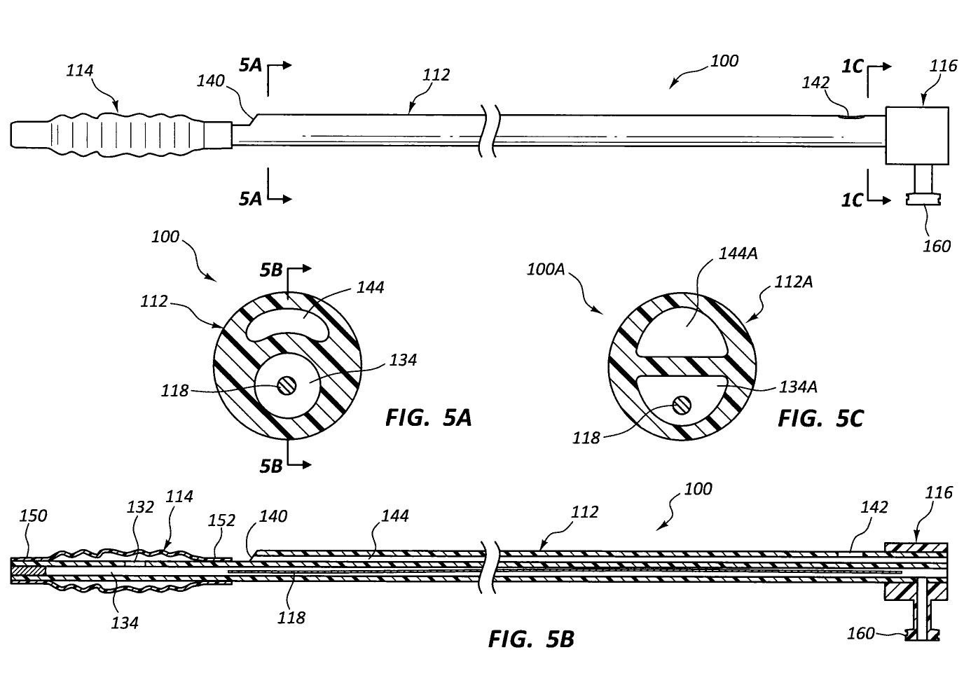

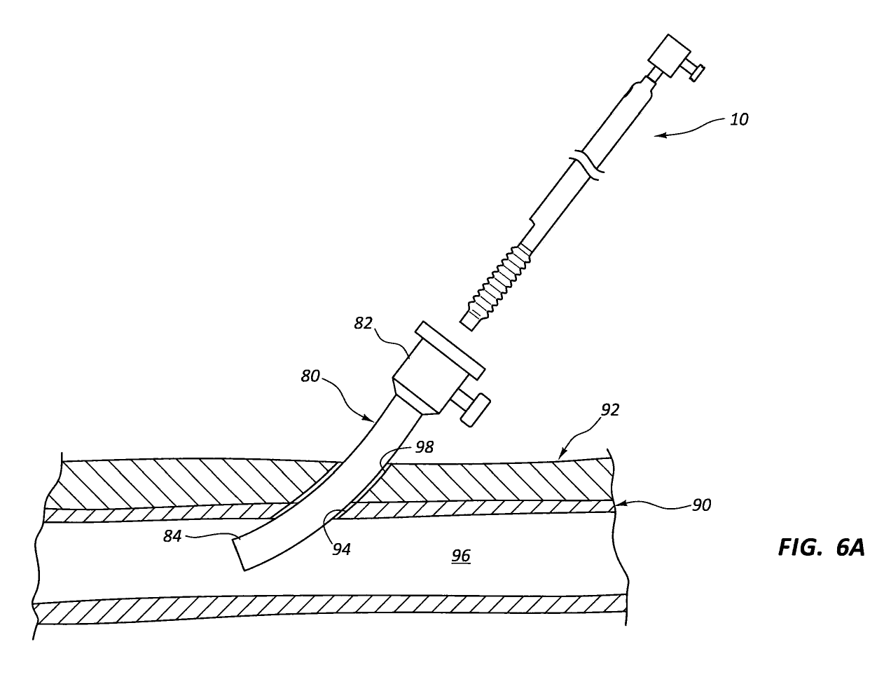

Referring now to FIGS. 5-5C, another example vessel location device 100 is shown including a catheter 112, an expandable member 114, a hub 116, and a core wire 118 (see FIG. 5B). A vessel location device 100 includes a dual lumen catheter 112 rather than a catheter having two separate tubes that are connected together, as in the vessel location device 10 described above. The catheter 112 includes an inflation lumen 134 and a flashback lumen 144. The inflation lumen 134 is connected in flow communication with an inflation port 160 of the hub 116. The inflation lumen 134 includes an inflation outlet 132 arranged in flow communication with the expandable member 114. The flashback lumen 144 includes distal and proximal openings 140, 142. The distal opening 140 is positioned proximal of the expandable member 114.

The expandable member 114 may include distal and proximal waists 150, 152. The expandable member 114 may be connected to the catheter 112 at a location distal of the distal opening 140 of the flashback lumen 144. The distal and proximal waists 150, 152 may be bonded to the catheter 112 using, for example, a heat bonding technique or an adhesive. A distal end of the inflation lumen 134 may be closed such that the inflation fluid flowing through the inflation lumen 134 is directed to the expandable member 114.

The proximal opening 142 of the flashback lumen 144 may be positioned in a sidewall of the catheter 112. The proximal opening 142 may be positioned at a location distal of the hub 116. In other examples, the proximal opening 142 is positioned in the hub 116 or at a location proximal of the hub 116.

In at least some examples, the flashback lumens 44, 144 described herein may serve a dual purpose, wherein a first purpose is to provide a blood flashback as a visual indicator to the operator, and a second purpose is to provide a path for delivery of sealing material to a vessel puncture. The sealing material may be delivered through the flashback lumen 44, 144. Alternatively, a separate tube may be inserted through the flashback lumen 44, 144, wherein the separate tube provides delivery of a volume of sealing material to the vessel puncture. The sealing material may flow out of the distal opening 40, 140 of the vessel location device, or the separately inserted tube, at a location adjacent to the vessel puncture.

FIG. 5A shows the inflation lumen 134 having a circular cross-sectional shape and the flashback lumen 144 having a crescent shape. FIG. 5C shows another example vessel location device 100A having a catheter 112A with semi-circular shapes for the inflation lumen 134A and flashback lumen 144A. Many other cross-sectional shapes are possible for the inflation lumens and blood flashback lumens disclosed herein.

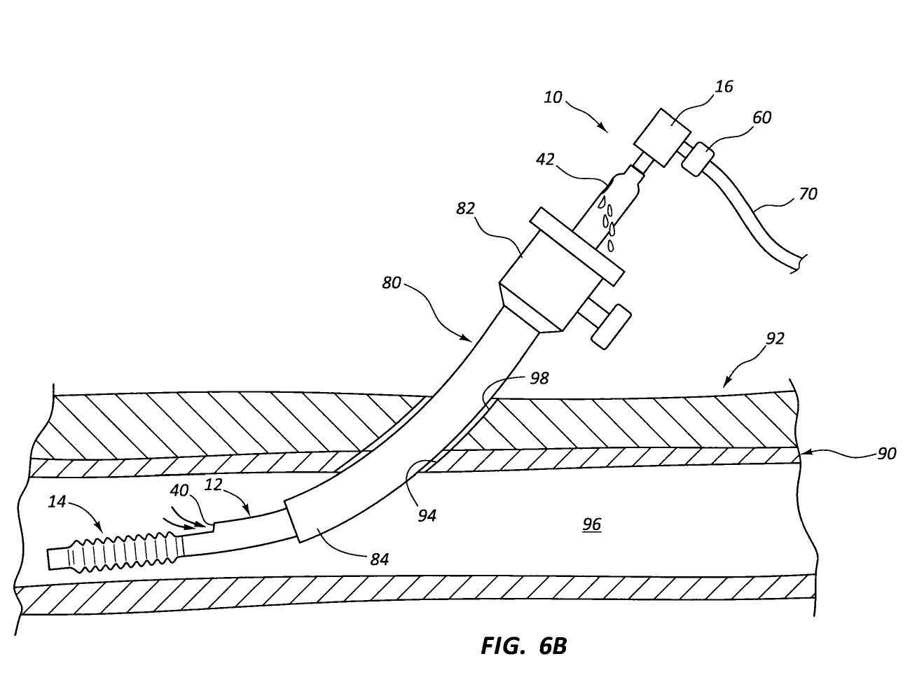

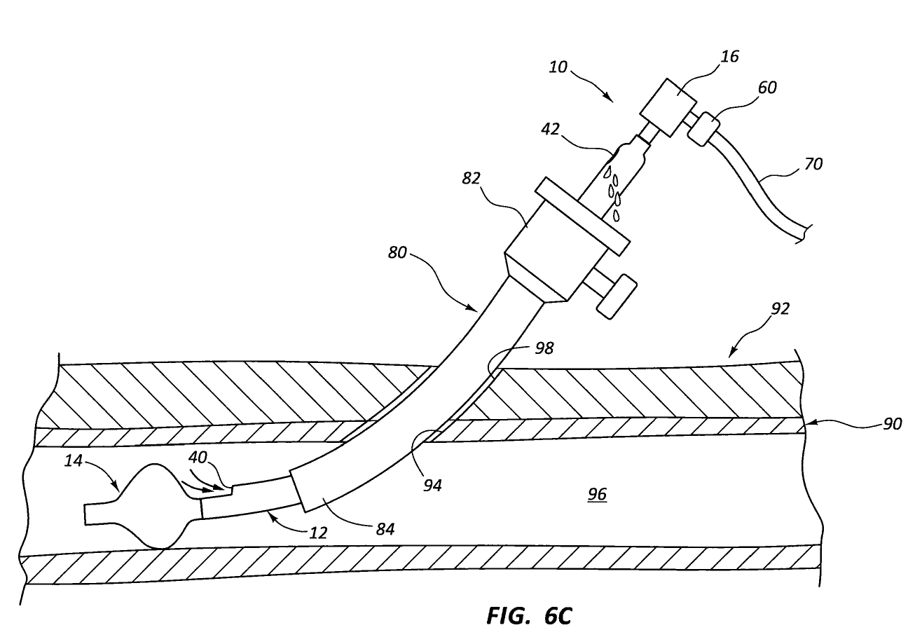

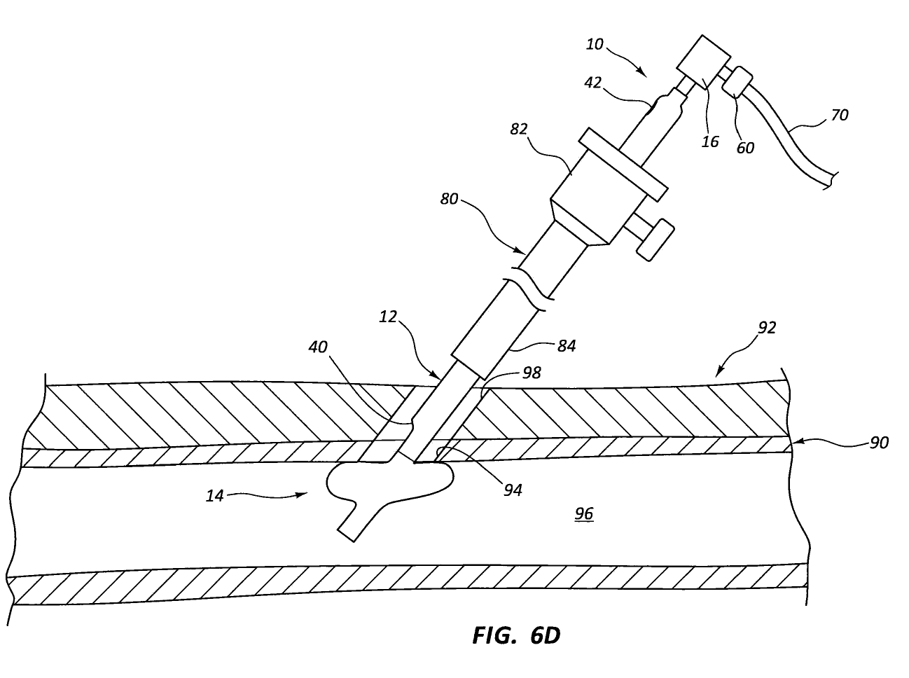

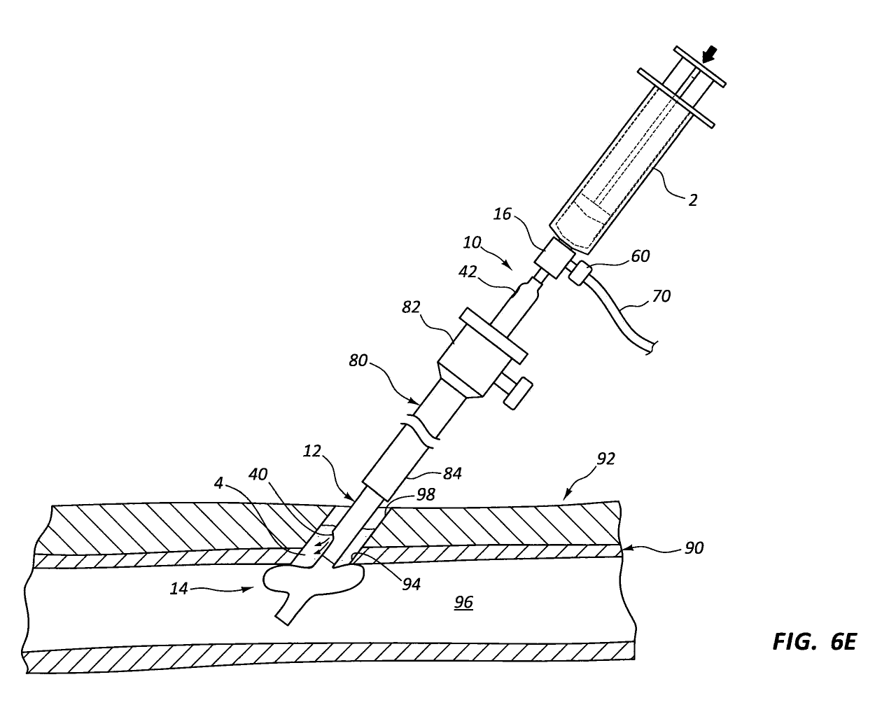

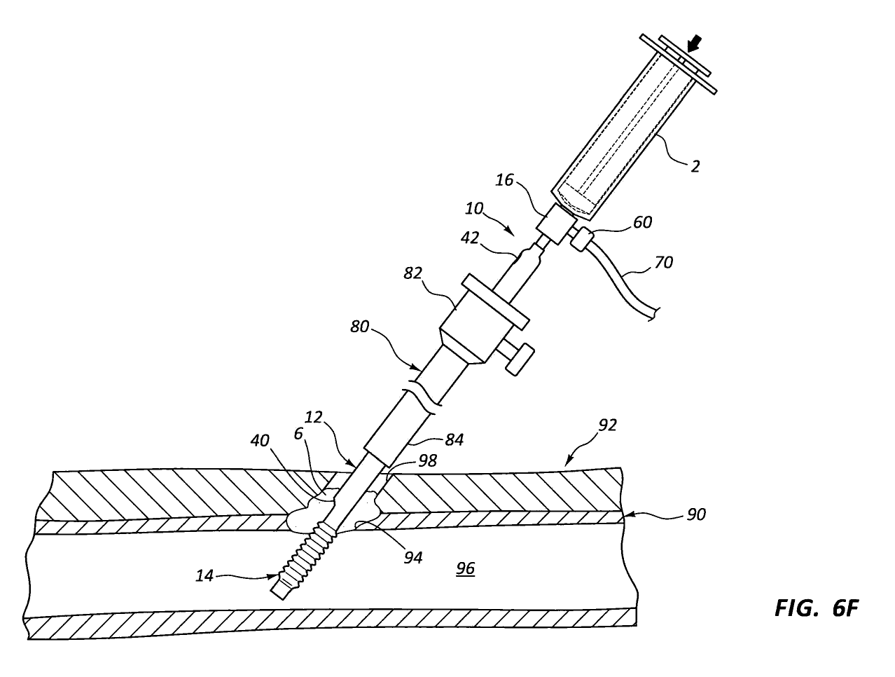

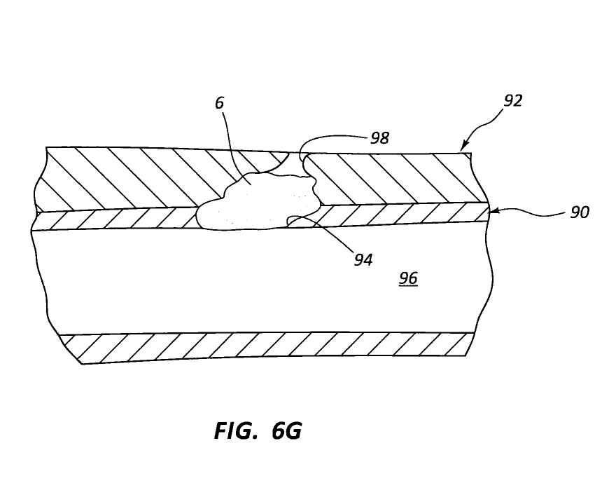

Referring now to FIGS. 6A-6G, an example method of locating a vessel puncture and then closing the vessel puncture using the vessel location device 10 is shown and described. FIG. 6A shows an insertion sheath 80 inserted through a vessel puncture 94 in a vessel 90 and an incision 98 of a tissue layer 92 to position a distal end 84 of the insertion sheath 80 within a vessel lumen 96. The insertion sheath 80 includes a hub 82 and a distal end 84. The vessel location device 10 is aligned with a proximal opening into the insertion sheath 80.

FIG. 6B shows the vessel location device 10 inserted through the insertion sheath 80 to position the distal opening 40 of the flashback lumen 44 and the expandable member 14 distal of the distal end 84 and within the vessel lumen 96. The insertion sheath 80 may be at least partially withdrawn to position the distal end 84 adjacent to or within the vessel puncture 94 and incision 98 prior to advancing the vessel location device 10 into the position shown in FIG. 6B. This partial withdrawal of the insertion sheath 80 may assist in avoiding unintentional advancement of the vessel location device 10 too far downstream into the vessel lumen 96. Blood flowing through the vessel lumen 96 may flow into the distal opening 40 as shown in FIG. 6B. The blood may flow through the flashback lumen 44 and out of the proximal opening 42 to provide a visual indicator to the operator that the expandable member 14 is positioned within the vessel lumen 96 and exposed to blood flow.

Referring to FIG. 6C, a volume of inflation fluid provided by an inflation source 70 may be delivered through the inflation lumen 34 to the expandable member 14. The expandable member 14 may expand into an expanded state as shown in 6E. Blood may continue to flow through the distal opening 40 and out of the proximal opening 42 to provide a visual indicator to the operator that the expandable member 14 is positioned within and exposed to blood flow in the vessel lumen 96.

The operator may then apply a withdrawal force to the vessel location device 10 and the insertion sheath 80 to abut the expandable member 14 against an inner surface of the vessel 90 adjacent to the vessel puncture 94 as shown in FIG. 6D. The expandable member 14 may provide at least temporary hemostasis for the vessel puncture 94. This temporary hemostatis may create cessation of blood flow through the vessel puncture 94 so that blood no longer flows through the flashback lumen 44. The cessation of blood flashback flow out of the proximal opening 42 provides a visual indicator to the operator that the expandable member 14 is properly seated and sealing the vessel puncture 94. The visual indication of the lack of blood flashback flow may be used in combination with tactile feedback (e.g., resistance to further withdrawing the vessel location device 10 in a proximal direction due to the abutting arrangement of the expandable member 14 against inner surface of the vessel 90) to assist the operator in confirming location of the vessel puncture 94. Other visual indicators may be provided on the vessel location device 10 such as, for example, markings along a length of the catheter 12 that show an approximate distance from the expandable member 14 or a depth of the vessel puncture 94 from an outer surface of the patient. Other markings may be provided that show relative position between the insertion sheath 80 and features of vessel location device 10.

The operator, having located and temporarily sealed the vessel puncture 94, may use the vessel location device 10 in various ways to further treat the patient. In one example, the insertion sheath 80 is further withdrawn along a length of the vessel location device 10 and a sealant delivery device may be advanced through the incision 98 to deliver sealing material to the vessel puncture 94. The sealant delivery device may connect to or track along the catheter 12 to a position adjacent to the vessel puncture 94. In another example, a separate sealant delivery device is advanced through the insertion sheath 80 to a position adjacent to the vessel puncture 94 for delivery of a sealing material.

In a still further example, sealing material is delivered through the vessel location device 10 to the vessel puncture 94. FIG. 6E shows a sealing material source 2 connected to the hub 16 and operated to deliver a volume of sealing material 4 to the vessel puncture 94. In one example, the sealing material 4 may be delivered through the flashback lumen 44. In other examples, the vessel location device 10 may include an additional lumen used solely for delivering the sealing material to the vessel puncture.

FIG. 6E shows the sealing material 4 being ejected through the distal opening 40 of the flashback lumen 44. The sealing material 4 may fill at least portions of the vessel puncture 94 and incision 98. In some examples, the sealing material source 2 may include a tube, which provides an additional sealing material lumen. The tube may be inserted through the flashback lumen 44 and out through the distal opening 40. The tube may traverse the proximal opening 42 such that sealing material is not inadvertently ejected through the proximal opening 42.

After the sealing material 4 is delivered, the sealing material 4 may be allowed to cure to form a sealing plug 6. The sealing plug 6 may have sufficient shape and structure to avoid inadvertently advancing into the vessel lumen 96 after the expandable member 14 has been operated into an unexpanded state as shown in FIG. 6F. The operator may then apply a withdrawal force to the vessel location device 10 to remove the vessel location device 10 proximally through the sealing plug 6. The sealing plug 6 may be bonded directly to portions of the vessel 90 and tissue layer 92.

After removal of the vessel location device 10, a channel or tract may remain formed through the sealing plug 6 upon removal of the vessel location device 10. The channel may be filled by further expansion of the sealing material 4. Alternatively, an additional volume of sealing material may be injected into the incision 98 using the vessel location device 10 or other sealant delivery device. This additional volume of sealing material may seal the channel in the sealing plug 6. FIG. 6G shows the sealing plug 6 in position after removal of the vessel location device 10.

While this invention has been described with reference to certain specific embodiments and examples, it will be recognized by those skilled in the art that many variations are possible without departing from the scope and spirit of this invention. The invention, as defined by the claims, is intended to cover all changes and modifications of the invention which do not depart from the spirit of the invention. The words “including” and “having,” as used in the specification, including the claims, shall have the same meaning as the word “comprising.”

What is claimed is:

1. A vessel location device, comprising:
a catheter having a distal end portion, a proximal end portion and a blood flashback lumen, the blood flashback lumen having a distal opening positioned at the distal end portion and a proximal opening at the proximal end portion;
an expandable member positioned at the distal end portion at a location distal of the distal opening of the blood flashback lumen;
an inflation member configured to deliver a flow of inflation fluid to the expandable member to selectively expand the expandable member within a vessel to temporarily seal a vessel puncture.

2. The vessel location device of claim 1, wherein the inflation member comprises an inflation tube extending through the blood flashback lumen, the expandable member being mounted to a distal end portion of the inflation tube.

3. The vessel location device of claim 1, wherein the catheter and inflation member are connected to each other at the distal end portion of the catheter.

4. The vessel location device of claim 1, wherein the catheter and inflation member are connected to each other at the proximal end portion of the catheter.

5. The vessel location device of claim 1, wherein the catheter and inflation member are connected to each other at a proximal bond location, and the proximal opening of the blood flashback lumen is positioned distal of the proximal bond location.

6. The vessel location device of claim 1, wherein the catheter comprises a dual lumen construction having first and second lumens, the blood flashback lumen including the first lumen and the inflation member including the second lumen.

7. The vessel location device of claim 6, wherein the first lumen has a circular cross-sectional shape and the second lumen has a non-circular cross-sectional shape.

8. The vessel location device of claim 6, wherein the first lumen has a non-circular cross-sectional shape and the second lumen has a circular cross-sectional shape.

9. The vessel location device of claim 1, wherein the inflation member comprises an inflation lumen and the inflation member is arranged coaxial with the blood flashback lumen.

10. A vessel location device, comprising:
a catheter having first and second lumens, a distal end portion and a proximal end portion;
an expandable member positioned at the distal end portion and arranged in flow communication with the first lumen, the second lumen having a distal opening arranged proximal of the expandable member at the distal end portion, and a proximal opening at the proximal end portion;
wherein the vessel location device is configured to advance through a vessel puncture and into a vessel where the expandable member is expanded to limit blood flow through the vessel puncture, and the second lumen provides blood flashback to indicate a position of the vessel location device relative to blood flow in the vessel.

11. The vessel location device of claim 10, wherein the catheter comprises first and second tubes defining the first and second lumens.

12. The vessel location device of claim 11, wherein the first and second tubes are bonded to each other at the distal and proximal end portions of the catheter.

13. The vessel location device of claim 11, wherein the second tube extends through the first lumen.

14. The vessel location device of claim 11, wherein the expandable member comprises an inflation balloon having a distal waist and a proximal waist, the inflation balloon being connected to the catheter at a distal bond formed between the proximal waist and the first and second tubes.

15. The vessel location device of claim 11, wherein the first and second tubes are connected together at a proximal bond, the proximal opening of the second lumen being positioned distal of the proximal bond.

16. The vessel location device of claim 10, further comprising a support core wire extending through one of the first and second lumens to the distal end portion.

17. The vessel location device of claim 10, wherein the catheter comprises an integrally formed, dual lumen construction.

18. A method of locating a vessel, comprising:
providing a catheter having a distal end portion, a proximal end portion, a blood flashback lumen, an inflation lumen, and an expandable member positioned at the distal end portion at a location distal of a distal opening of the blood flashback lumen;
positioning the expandable member through a vessel puncture;
providing a flow of blood through the blood flashback lumen;
inflating the expandable member via the inflation lumen;
abutting the inflated expandable member into contact with an inner surface of the vessel adjacent to the vessel puncture to temporarily seal the vessel puncture;
wherein temporarily sealing the vessel puncture stops blood flow through the blood flashback lumen.

19. The method of claim 18, wherein the expandable member comprises an inflation balloon having a distal waist and a proximal waist, and the catheter comprises first and second tubes defining the inflation lumen and blood flashback lumen, respectively, the method further comprising providing a distal bond between the proximal waist and first and second tubes.

20. The method of claim 18, wherein providing a flow of blood through the blood flashback lumen includes flowing blood into the distal opening of the blood flashback lumen, and flowing blood out of a proximal opening of the blood flashback lumen at a location distal of a proximal end of the inflation lumen.

21. The method of claim 18, wherein providing the catheter comprises forming the catheter as an extruded dual lumen structure.
